(19) United States
(12) Patent Application Publication
(10) Pub. No.: US 2022/0079975 A1
(43) Pub. Date: Mar. 17, 2022

(54) METHOD FOR MITIGATION OF NON-ALCOHOLIC FATTY LIVER DISEASE BY USE OF A COMPOSITION COMPRISING SMALL-MOLECULE FUCOIDAN AND FUCOXANTHIN

(71) Applicants: HI-Q MARINE BIOTECH INTERNATIONAL LTD., TAIPEI CITY (TW); TAIPEI MEDICAL UNIVERSITY, TAIPEI CITY (TW); Taipei Municipal Wanfang Hospital (Managed by Taipei Medical University), Taipei City (TW)

(72) Inventor: Ming-Shun WU, Taipei City (TW)

(21) Appl. No.: 17/017,937

(22) Filed: Sep. 11, 2020

(51) Int. Cl. A61K 31/737; A61K 31/336; A61K 9/00; A61P 1/16

(52) U.S. Cl. CPC A61K 31/737; A61K 31/336; A61P 1/16; A61K 9/0019; A61K 9/0053

(57) ABSTRACT

The present invention relates to a method for mitigation of non-alcoholic fatty liver disease, the method comprising administering an effective amount of a composition comprising small-molecule fucoidan and fucoxanthin to a subject in need thereof, and achieves the efficacy by means of improving the controlled attenuation parameter and blood biochemical parameters such as alanine aminotransferase (ALT), glucose ante cibum (AC) and glycated hemoglobin (HbA1c).

### BACKGROUND OF THE INVENTION

### 1. Field of the Invention

The present invention relates to a method for mitigation of non-alcoholic fatty liver disease, especially by use of a composition comprising small-molecule fucoidan and fucoxanthin.

### 2. Description of the Prior Arts

Clinically, non-alcoholic fatty liver disease and alcoholic fatty liver disease are two subtypes of fatty liver disease, and the clinical definition of fatty liver disease is that intrahepatic fat makes up at least 5% of liver weight, or that degenerative fat vacuoles are present in at least ten percent of the hepatocytes in the liver biopsy. Nowadays, fatty liver disease is one of the diseases of modern civilization, and has been regarded as one of the major liver diseases in most of the developed countries, such as Europe and the United States. While non-alcoholic fatty liver disease and alcoholic fatty liver disease are distinguished clinically and have different causes of diseases, they share the same pathological features, such as steatosis, steatohepatitis, liver fibrosis, or even liver cirrhosis.

While alcoholic fatty liver disease is caused by long-term consumption of alcohol with an amount more than 50 grams of alcohol per day, non-alcoholic fatty liver disease is more common in patients having lipid metabolism disorders, diabetes and obesity, etc. along with the symptoms of hyperlipidemia and hyperglycemia, etc. Further, clinical studies indicate that diabetic patients or obese people with BMI higher than normal are high risk groups of non-alcoholic fatty liver disease. When the disorder of non-alcoholic fatty liver disease continues, it may develop into non-alcoholic steatohepatitis (NASH) with high probability according to the normal disease progression, and non-alcoholic steatohepatitis plays an important role in liver fibrosis. So far, the mechanism and causes of these diseases are yet to be clear, but studies show that patients with both diabetes and fatty liver disease have a higher chance to develop non-alcoholic steatohepatitis than patients with fatty liver disease only.

Researches on non-alcoholic fatty liver disease are in full swing around the world, and many hypotheses and possible mechanisms are continuously updated. Further, while several possible treatments are proposed as cell and animal experiments, they are rarely adopted in clinical trials. In current technologies, the approved diabetes drug, Pioglitazone, shows the efficacy to improve liver function along with the risk of weight gain and bladder cancer in clinical trials. Although the drug, Liraglutide, could be used to lose weight, the administration by subcutaneous injection is required. Hence, the treatment for non-alcoholic fatty liver disease without toxicity in humans are yet to be developed.

### SUMMARY OF THE INVENTION

In order to provide a method for mitigation of non-alcoholic fatty liver disease that is best suitable for humans and without toxicity, the present invention intends to provide a composition comprising small-molecule fucoidan and fucoxanthin for mitigation of non-alcoholic fatty liver disease.

In order to achieve the above object, the present invention provides a method for mitigation of non-alcoholic fatty liver disease, comprising administering an effective amount of a composition comprising small-molecule fucoidan and fucoxanthin to a subject in need thereof.

Preferably, the composition consists of small-molecule fucoidan and fucoxanthin.

Preferably, the composition aforementioned may be prepared as a medicament, a medical food or a health food.

The medical food means a food which is formulated to be consumed or administered enterally under the supervision of a physician and which is intended for the specific dietary management of a disease or condition for which distinctive nutritional requirements, based on recognized scientific principles, are established by medical evaluation.

The health food is a food with specific ingredients, which adjusts the physiological function and improves health and could also be referred to as functional food or dietary supplements.

Preferably, the composition could be in various forms including, but not limited to, liquid, semi-solid and solid dosage forms, such as a solution, an emulsion, a suspension, a patch, a liniment, an aerosol, a paste, a foam, a drop, a salve, a powder, a tablet, a pill, a lozenge, a troche, a chewing gum, a capsule, a liposome, an injection, and other similar or applicable dosage forms.

More preferably, the composition is in an enteral or a parenteral dosage form, wherein the enteral dosage form is an oral dosage form, and the oral dosage form is a solution, a suspension, a tablet or a capsule. The parenteral dosage form is an injection.

Preferably, the composition is prepared as a medical food or a health food, and the medical food or health food is in various forms including, but not limited to, liquid, semi-solid and solid dosage forms, such as a solution, an emulsion, a suspension, a powder, a tablet, a pill, a lozenge, a troche, a chewing gum, a capsule, a liposome, and other similar or applicable dosage forms.

Preferably, the composition is prepared as a medicament, and the medicament comprises the composition comprising small-molecule fucoidan and fucoxanthin and a pharmaceutical acceptable carrier.

Preferably, the small-molecule fucoidan has a molecular weight ranging from about 400 Daltons to 100,000 Daltons; more preferably, the small-molecule fucoidan has a molecular weight ranging from about 400 Daltons to 500, 600, 700, 800, 900, 1,000, 1,500, 2,000, 5,000, 10,000, 20,000, 30,000 or 50,000 Daltons, the small-molecule fucoidan has an average molecular weight ranging from about 400 Daltons to 2,000 Daltons; more preferably, from 500 Daltons to 600, 700, 800, 900 or 1,000 Daltons; in one embodiment the small-molecule fucoidan has a unit shown as follows:

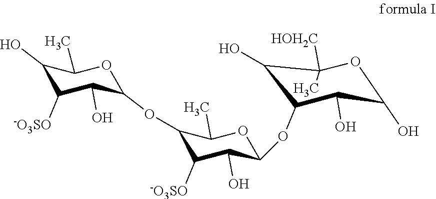

Preferably, the small-molecule fucoidan is extracted from algae selected from the group consisting of Phaeophyceae. The small-molecule fucoidan is commercially available, and could be obtained by extracting a variety of brown algae comprising, but not limited to, common edible algae, such as kelps, undaria pinnitafida, horsetail kelps, kombu and fucus, etc.

In one embodiment, fucoxanthin has a molecular formula of C₄₂H₅₈O₆, and has a structure similar to β-carotene or vitamin A. Fucoxanthin is classified as carotenoids, and is a common pigment in the chloroplast of Phaeophyceae. Fucoxanthin is commercially available or may be obtained by extraction from the brown algae aforementioned. Preferably, fucoxanthin is the fucoxanthin sold by Hi-Q Marine Biotech Co., Ltd.

Preferably, in the composition comprising small-molecule fucoidan and fucoxanthin, a weight ratio of the small-molecule fucoidan to the fucoxanthin ranges from 5:1 to 1:5; more preferably, the weight ratio of the small-molecule fucoidan to the fucoxanthin in the composition ranges from 1:1 to 1:5.

Preferably, an effective amount of the small-molecule fucoidan ranges from 0.4 g to 2 g per day, and an effective amount of the fucoxanthin ranges from 0.4 g to 2 g per day; more preferably, the effective amount of the small-molecule fucoidan is 1.65 g per day, and the effective amount of the fucoxanthin is 1.65 g per day.

Preferably, the effective amount of the composition is 0.8 g to 4 g per day; more preferably, the effective amount of the composition is 3.3 g per day.

Preferably, the effective amount aforementioned is the effective dose for humans.

Preferably, the mitigation of non-alcoholic fatty liver disease comprises: reducing the controlled attenuation parameter; reducing liver stiffness; reducing a blood biochemical parameter, and the blood biochemical parameter is selected from the group consisting of alanine aminotransferase (ALT), glucose ante cibum (AC) and glycated hemoglobin (HbA1c); or reducing insulin resistance.

The advantage of the present invention is to achieve the mitigation of non-alcoholic fatty liver disease through the composition comprising small-molecule fucoidan and fucoxanthin of the present invention. The composition comprising small-molecule fucoidan and fucoxanthin of the present invention is further used for improving the expression level of serum alanine aminotransferase, reducing the controlled attenuation parameter, reducing glycated hemoglobin and reducing insulin resistance.

### BRIEF DESCRIPTION OF THE DRAWINGS

FIG. 1 is a time series chart showing the change of the controlled attenuation parameter in the experimental group and the control group. The vertical axis pinpoints the change of the controlled attenuation parameter and the unit is dB/m. The horizontal axis is the increments of time and the unit is month.

FIG. 2 is a time series chart showing the change of serum alanine aminotransferase in the experimental group and the control group. The vertical axis pinpoints the change of serum alanine aminotransferase (ALT) and the unit is U/L. The horizontal axis is the increments of time and the unit is month.

FIG. 3 is a time series chart showing the change of glycated hemoglobin in the experimental group and the control group. The vertical axis pinpoints the change of glycated hemoglobin (HbA1c) and the unit is %. The horizontal axis is the increments of time and the unit is month.

FIG. 4 is a time series chart showing the change of glucose ante cibum in the experimental group and the control group. The vertical axis pinpoints the change of glucose ante cibum (AC) and the unit is mmol/L. The horizontal axis is the increments of time and the unit is month.

FIG. 5 is a time series chart showing the change of insulin in the experimental group and the control group. The vertical axis pinpoints the change of insulin and the unit is μIU/mL. The horizontal axis is the increments of time and the unit is month.

FIG. 6 is a time series chart showing the change of triglyceride in the experimental group and the control group. The vertical axis pinpoints the change of triglyceride (TG) and the unit is mg/dL. The horizontal axis is the increments of time and the unit is month.

FIG. 7 is a time series chart showing the change of leptin in the experimental group and the control group. The vertical axis pinpoints the change of leptin and the unit is μg/L. The horizontal axis is the increments of time and the unit is month.

FIG. 8 is a time series chart showing the change of adiponectin in the experimental group and the control group. The vertical axis pinpoints the change of adiponectin and the unit is ng/mL. The horizontal axis is the increments of time and the unit is month.

### DETAILED DESCRIPTION OF THE PREFERRED EMBODIMENTS

The present invention will be further illustrated by the following examples, and these examples shall not be interpreted as a limitation of the content of the present invention aforementioned. A person having ordinary skill in the art of the present invention may make some improvements and modifications without deviating from the scope of the present invention.

### Preparation Example 1: Preparation of a Composition Comprising Small-Molecule Fucoidan and Fucoxanthin

The purpose of this preparation example is to prepare a composition comprising small-molecule fucoidan and fucoxanthin. The preparation method is as follows: raw materials of brown algae were firstly washed along with the reduction of heavy metals therein, extracted to obtain small-molecule fucoidan and fucoxanthin respectively according to the conventional methods, and then the small-molecule fucoidan and fucoxanthin were sterilized to obtain semi-finished products, which were then dried and granulated. A composition 550 mg of small-molecule fucoidan and fucoxanthin were filled into a capsule to obtain a composition comprising small-molecule fucoidan and fucoxanthin, wherein the weight ratio of the small-molecule fucoidan to the fucoxanthin is 1:1. The capsule comprising small-molecule fucoidan and fucoxanthin was provided by Hi-Q Marine Biotech Co., Ltd., and the small-molecule fucoidan has a molecular weight ranging from 500 Daltons to 1000 Daltons.

### Example 1: Administration of a Composition Comprising Small-Molecule Fucoidan and Fucoxanthin to Subjects and Measurement of Fatty Liver, Liver Fibrosis and Metabolism Indexes

The subjects were patients diagnosed to have fatty liver disease by abdominal ultrasound and further classified to have non-alcoholic fatty liver disease, along with the condition that the patients were not taking vitamin E, Pioglitazone and Liraglutide injection at that time. The subjects are randomly divided into two groups in a double-blind test. The first group is the experimental group (EG), and was administered with the capsule comprising small-molecule fucoidan and fucoxanthin prepared in Preparation Example 1. The experiment lasted for 6 months, and the subjects took 6 capsules a day in a manner of 3 capsules in the morning and 3 capsules at night, and each capsule contains 275 mg of small-molecule fucoidan and 275 mg of fucoxanthin. The second group is the control group (CG), and was administered with placebo capsules filled with cellulose. The experiment also lasted for 6 months, and the subjects took 6 capsules a day in a manner of 3 capsules in the morning and 3 capsules at night. The subjects in the experimental group and the control group were tested for creatinine (Cr), glucose ante cibum (AC) and liver function index including alanine transaminase (ALT), aspartate transaminase (AST), and fibroScan values for the evaluation of fatty liver disease and liver fibrosis before the experiment started as the first test, and at full 1 month, 2 months, 3 months, 4 months and 6 months of the experiment. Further, the subjects in both groups were tested for uric acid (UA) and blood lipids including total cholesterol, high-density lipoprotein (HDL), low-density lipoprotein (LDL), triglyceride (TG), adiponectin, leptin and insulin at the first test, and at full 1 month, 3 months and 6 months of the experiment. Also, the subjects in both groups were tested for glycated hemoglobin (HbA1c) at the first test, and at full 3 months and 6 months of the experiment.

### Example 2: The Result of the First Test

According to the experimental method of Example 1, the monitored items of age, body mass index (BMI), glucose ante cibum (AC), total cholesterol, high-density lipoprotein, low-density lipoprotein, uric acid, creatinine, aspartate aminotransferase, alanine aminotransferase, liver stiffness, controlled attenuation parameter, glycated hemoglobin, triglyceride, adiponectin, leptin, pancreatic 13 cells and insulin of the subjects in both groups are recorded at the first test to obtain a data of the first test (R1). As shown in Table 1, the data of the controlled attenuation parameter indicates that the fatty liver disease of the experimental group is slightly more severe than that of the control group, but the test result after the completion of the experiments shows that the experimental group has better improvement effect than the control group. For the rest of the data in Table 1, no statistically significant difference is identified so as to establish a randomized experiment.

TABLE 1
The data of fatty liver disease, liver fibrosis and metabolism indexes
of the experimental group (EG) and control group (CG) at the first test

| Monitored items | EG | CG | P value |
|---|---|---|---|
| Age | 55 ± 12.5 | 59 ± 10.5 | 0.23 |
| Body mass index (BMI) | 28.7 ± 3.9 | 27.5 ± 4.0 | 0.31 |
| Glucose ante cibum (AC) | 97.8 ± 17.3 | 103.2 ± 14.7 | 0.29 |
| Insulin | 10.3 ± 4.1 | 11.1 ± 5.1 | 0.58 |
| Total Cholesterol | 190.3 ± 39.4 | 198.9 ± 26.6 | 0.41 |
| HDL | 50.5 ± 9.4 | 50.2 ± 9.9 | 0.91 |
| LDL | 119.3 ± 30.9 | 125 ± 20.5 | 0.49 |
| Triglyceride | 161.7 ± 80 | 163.4 ± 75.7 | 0.99 |
| Adiponectin | 5652 ± 2433.3 | 6486 ± 3842.7 | 0.68 |
| Leptin | 21.8 ± 12.4 | 24.1 ± 10.6 | 0.54 |
| Uric acid (UA) | 5.9 ± 1.2 | 5.6 ± 0.8 | 0.36 |
| Creatinine (Cr) | 0.74 ± 0.1 | 0.72 ± 0.2 | 0.71 |
| AST | 28.1 ± 7.0 | 28.5 ± 15.9 | 0.33 |
| ALT | 42.2 ± 17.2 | 33.5 ± 21.7 | 0.16 |
| Liver Stiffness | 6.5 ± 1.8 | 6.7 ± 2.9 | 0.52 |
| CAP | 343.6 ± 44.6 | 303.6 ± 66.9 | 0.03* |

### Example 3: The Effect of the Composition Comprising Small-Molecule Fucoidan and Fucoxanthin on the Fatty Liver Disease, Liver Fibrosis and Metabolism Indexes of the Subjects at Full 3 Months of the Experiment

The capsules comprising small-molecule fucoidan and fucoxanthin as described in Preparation Example 1 were given to the subjects of the experimental group in Example 1, and the data of fatty liver disease, liver fibrosis and metabolism indexes of the subjects at full 3 months of the experiment are analyzed. The data of the first test (R1) are subtracted from the data at full 3 months (R2) to obtain a first set of differences (D1), and the first set of differences (D1) are the changes of the monitored items at full 3 months, wherein the data of the first test (R1) are the values recorded in Example 2 and shown in Table 1; and the mean value and standard deviation of the change of the experimental group and the control group at full 3 months of the experiment are shown respectively in Table 2. The effective improvement rates (%) at full 3 months for each monitored item are also shown in Table 2. Meanwhile, the data of the change of fatty liver disease, liver fibrosis and metabolism indexes of the subjects at full 6 months of the experiment are shown in Table 3. The effective improvement rates of liver stiffness, insulin and insulin resistance at full 3 months and 6 months of the experiment are shown in Table 4.

TABLE 2
The data of the change of fatty liver disease, liver fibrosis and
metabolism indexes of the experimental group (EG) and control
group (CG) and the effective improvement rate thereof at full 3
months of the experiment

| | | |
|---|---|---|
| Mean value of the change of | Standard | Effective improvement |
| the monitored items | deviation | rates (%) |

Alanine aminotransferase (U/L)

| | | | |
|---|---|---|---|
| EG | −6.952 | 9.362 | 85.7 |
| CG | −2.842 | 9.002 | p = 0.024 |

Controlled attenuation parameter (CAP) (dB/m)

| | | | |
|---|---|---|---|
| EG | −16.95 | 42.529 | 57.1 |
| CG | 5.579 | 49.302 | p = 0.243 |

Glycated hemoglobin (HbA1c) (%)

| | | |
|---|---|---|
| EG | −0.033 | 0.114 |
| CG | 0.069 | 0.289 |

Pancreatic β cells (%)

| | | | |
|---|---|---|---|
| EG | −30.74 | 174.05 | 52.4 |
| CG | 8.674 | 53.321 | |

TABLE 3
The data of the change of fatty liver disease, liver fibrosis and
metabolism indexes of the experimental group (EG) and control
group (CG) and the effective improvement rate thereof at full
6 months of the experiment.

| | | |
|---|---|---|
| Mean value of the change of | Standard | Effective improvement |
| the monitored items | deviation | rates (%) |

Alanine aminotransferase (U/L)

| | | | |
|---|---|---|---|
| EG | −5.571 | 10.438 | 76.2 |
| CG | 2.737 | 9.752 | p = 0.001 |

Controlled attenuation parameter (CAP) (dB/m)

| | | | |
|---|---|---|---|
| EG | −26.62 | 25.841 | 85.7 |
| CG | 8.579 | 45.002 | p = 0.011 |

Glycated hemoglobin (HbA1c) (%)

| | | | |
|---|---|---|---|
| EG | −0.049 | 0.123 | 67 |
| CG | 0.086 | 0.292 | p = 0.004 |

Pancreatic β cells (%)

| | | | |
|---|---|---|---|
| EG | −18.74 | 195.423 | 81 |
| CG | 29.95 | 87.102 | |

TABLE 4
The effective improvement rate of liver stiffness, insulin and insulin
resistance at full 3 months and 6 months of the experimental group.

| | Liver stiffness | Insulin | Insulin |
|---|---|---|---|
| | (Kpa) | (μIU/mL) | resistance |
| The effective improvement | 66.7 | 47.6 | 47.6 |
| rate at full 3 months | | | |
| The effective improvement | 61.9 | 23.8 | 19 |
| rate at full 6 months | | | |

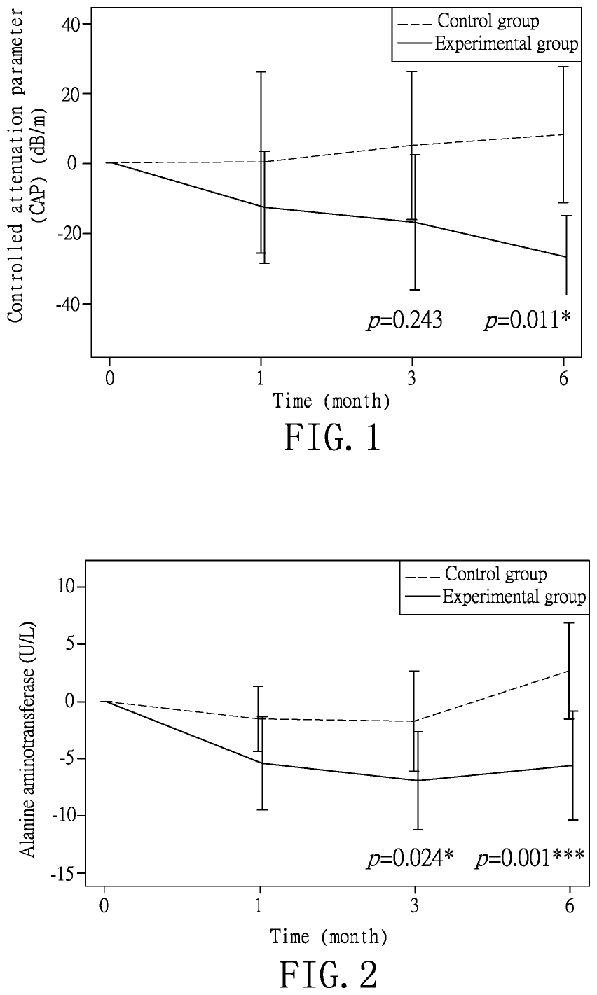

The mean value of the change of controlled attenuation parameter (CAP) of the experimental group at full 3 months of the experiment is −16.95±42.529 dB/m. Compared with that of the control group, the effective improvement rate is 57.1%. The mean value of the change of controlled attenuation parameter (CAP) of the experimental group at full 6 months of the experiment is −26.62±25.841 dB/m. Compared with that of the control group, the effective improvement rate is 85.7%. Further, FIG. 1 shows a reduced tendency of the controlled attenuation parameter at full 1 month and 3 months in the experimental group in comparison with that in the control group, and a statistically significant difference is identified in the experimental group at full 6 months of the experiment in comparison with that in the control group.

The mean value of the change of alanine aminotransferase of the experimental group at full 3 months of the experiment is −6.952±9.362 U/L. Compared with that of the control group, the effective improvement rate is 85.7%. The mean value of the change of alanine aminotransferase of the experimental group at full 6 months of the experiment is −5.571±10.438 U/L. Compared with that of the control group, the effective improvement rate is 76.2%. Further, FIG. 2 shows a reduced tendency of alanine aminotransferase at full 1 month in the experimental group in comparison with that in the control group, and a statistically significant difference is identified in the experimental group at full 3 months and 6 months of the experiment respectively in comparison with that in the control group.

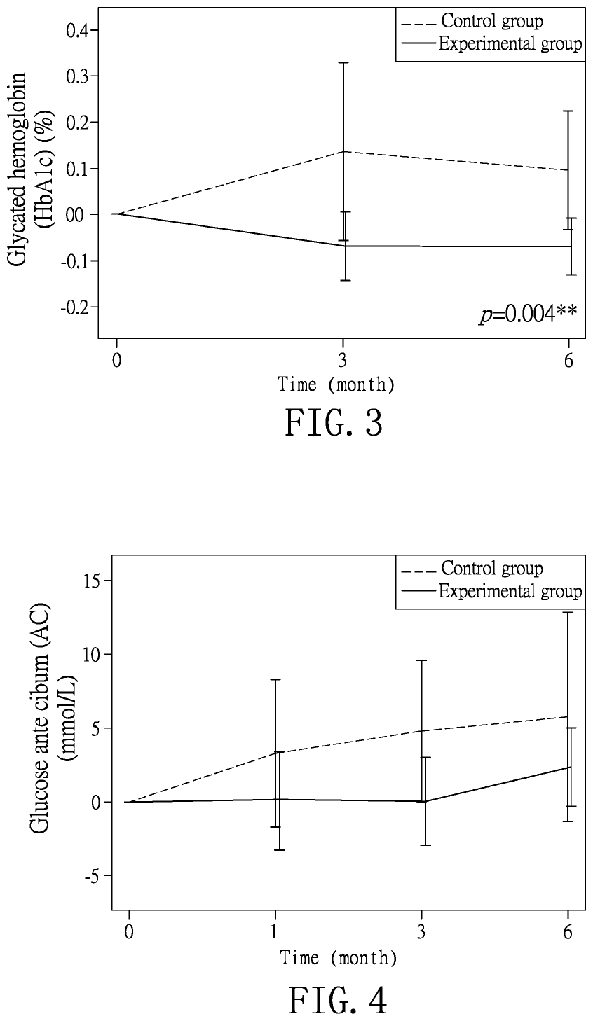

The mean value of the change of glycated hemoglobin (HbA1c) of the experimental group at full 3 months of the experiment is −0.033±0.114%. The mean value of the change of glycated hemoglobin (HbA1c) of the experimental group at full 6 months of the experiment is −0.049±0.123%. Further, FIG. 3 shows a reduced tendency of glycated hemoglobin at full 3 months of the experiment in the experimental group in comparison with that in the control group, and a statistically significant difference is identified in the experimental group at full 6 months of the experiment in comparison with that in the control group.

Table 2 shows that the mean value of the change of pancreatic β cells of the experimental group at full 3 months of the experiment is −30.74±174.05%. Compared with that of the control group, the effective improvement rate is 52.4%. Table 3 shows that the mean value of the change of pancreatic β cells of the experimental group at full 6 months of the experiment is −18.74±195.423%. Compared with that of the control group, the effective improvement rate is 81%.

Referring to the effective improvement rate of liver stiffness, insulin and insulin resistance of the experimental group at full 3 months and 6 months of the experiment in Table 4, the composition of the present invention also facilitates mitigation of the liver stiffness, insulin and insulin resistance of patients.

FIG. 4 shows a reduced tendency of glucose ante cibum (AC) at full 1 month, 3 months and 6 months of the experiment in the experimental group in comparison with that in the control group.

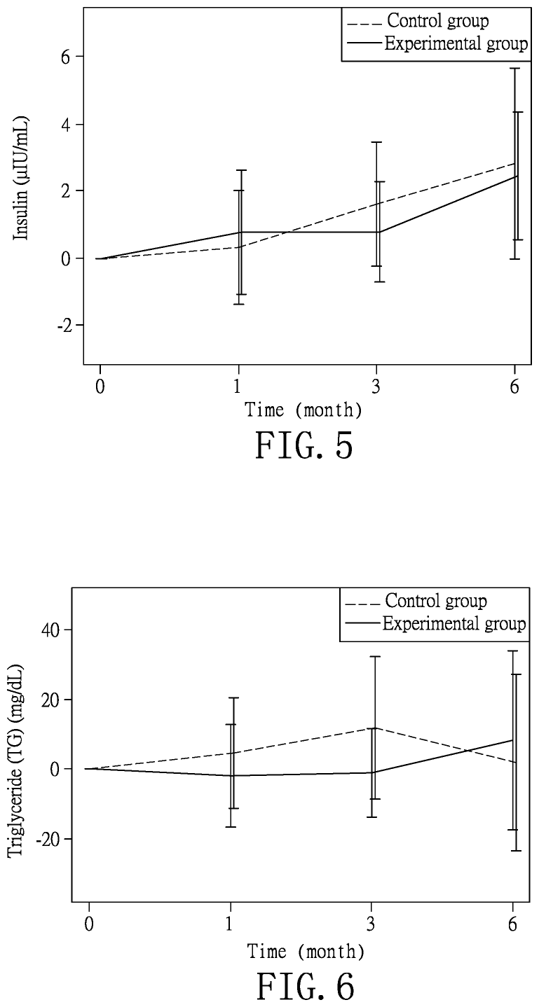

FIGS. 5 and 6 are the changes of insulin and triglyceride (TG) at full 1 month, 3 months and 6 months of the experiment in the experimental group respectively, and shows no tendency of improvement.

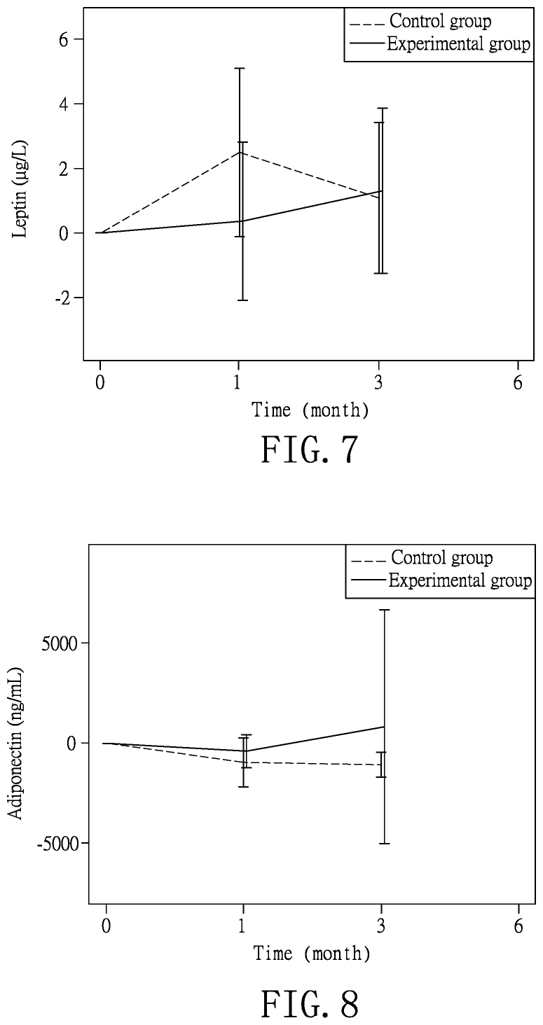

FIGS. 7 and 8 are the changes of leptin and adiponectin at full 1 month and 3 months of the experiment in the experimental group respectively, and shows no tendency of improvement.

One can use the data of the improvement of alanine aminotransferase, controlled attenuation parameter, glycated hemoglobin and pancreatic β cells to ascertain the efficacy for mitigating non-alcoholic fatty liver disease of patients. To sum up, the composition comprising small-molecule fucoidan and fucoxanthin could be used for mitigation of non-alcoholic fatty liver disease.

1. A method for mitigation of non-alcoholic fatty liver disease, comprising administering an effective amount of a composition comprising small-molecule fucoidan and fucoxanthin to a subject in need thereof, wherein the mitigation of non-alcoholic fatty liver disease comprises reducing liver stiffness, and the subject is 55 years old.

2. The method of claim 1, wherein the small-molecule fucoidan has a molecular weight ranging from 400 Daltons to 100,000 Daltons.

3. The method of claim 1, wherein the small-molecule fucoidan has an average molecular weight ranging from 400 Daltons to 2,000 Daltons.

4. The method of claim 1, wherein a weight ratio of the small-molecule fucoidan and the fucoxanthin in the composition ranges from 5:1 to 1:5.

5. The method of claim 4, wherein an effective amount of the small-molecule fucoidan ranges from 0.4 g to 2 g per day; and an effective amount of the fucoxanthin ranges from 0.4 g to 2 g per day.

6. The method of claim 1, wherein the composition is prepared as a medicament, a medical food or a health food.

7. The method of claim 1, wherein the composition is in an enteral or a parenteral dosage form.

8. The method of claim 7, wherein the enteral dosage form is an oral dosage form, and the oral dosage form is a solution, a suspension, a lozenge or a capsule.

9. The method of claim 7, wherein the parenteral dosage form is an injection.

10. The method of claim 1, wherein the mitigation of non-alcoholic fatty liver disease further comprises: reducing a controlled attenuation parameter; reducing a blood biochemical parameter selected from the group consisting of alanine aminotransferase (ALT), glucose ante cibum (AC) and glycated hemoglobin (HbA1c): or reducing insulin resistance.
